(19) United States
(12) Patent Application Publication
(10) Pub. No.: US 2006/0089669 A1
(43) Pub. Date: Apr. 27, 2006

(54) Implantation tool for adjustable implantable genitourinary device

(75) Inventors: Dale L. Schreiner, Cologne, MN (US); James E. Cabak, Plymouth, MN (US); Michael R. Satteson, St. Paul, MN (US); John H. Burton, Minnetonka, MN (US); Timothy C. Cook, Wayzata, MN (US)

Correspondence Address: Schwegman, Lundberg, Woessner & Kluth, P.A., P.O. Box 2938, Minneapolis, MN, 55402, US

(73) Assignee: Uromedica, Inc.

(21) Appl. No.: 11/226,519

(22) Filed: Sep. 14, 2005

Related U.S. Application Data
(60) Provisional application No. 60/609,640, filed on Sep. 14, 2004.

(51) Int. Cl. A61B 17/28

(52) U.S. Cl. 606/205

(57) ABSTRACT

The present application includes a system for the preparation of a patient for the implantation of post-operatively adjustable coaptation device. For example, the present subject matter includes a tool having a proximal portion and a distal portion, and an elongate section disposed between the proximal portion and the distal portion. The tool additionally includes jaws hinged at a first joint, the first joint located at the distal portion of the tool, the jaws adapted to vary between an open state and a closed state, the closed state defining a blunt tip. Also, the tool includes a handle located at the proximal portion of the tool. The handle and the jaws are connected so that motion of the handle moves the jaws from the closed state to the open state.

### RELATED APPLICATION

This application claims the benefit of U.S. Provisional Application Ser. No. 60/609,640 filed Sep. 14, 2004, which is incorporated by reference.

### TECHNICAL FIELD

This disclosure relates generally to surgical implements, and more particularly to a tool for implantation of a genitourinary device.

### BACKGROUND

Various implantable devices, such as inflatable/distensible medical devices, are available for implant in a patient to control flow through a body lumen. For example, inflatable devices intended for permanent genitourinary implantation are available to coapt a body lumen. Additionally, other forms of lumen coaptation are available, such as tissue bulking with injectable matter such as collagen.

Many devices are sized and implanted during surgery, and are not post-operatively adjustable. Because these devices are not post-operatively adjustable, performance loss caused by physiological changes is difficult to correct. For example, problems such as migration affect these designs. Additionally, in the case of injectable tissue bulking agent, bulking agent is absorbed or redistributed.

One cause of these phenomena is that intrusive surgical methods do not always reliably prepare body tissue for accepting an implantable coaptation system. Improved preparation could decrease instances of migration. What is needed is a system which is useful for the preparation of a patient for the implantation of post-operatively adjustable coaptation device.

### SUMMARY

The above-mentioned problems and others not expressly discussed herein are addressed by the present subject matter and will be understood by reading and studying this specification.

In varying embodiments, the present subject matter includes a tool for use with a sheath, comprising an elongate section disposed between a proximal portion of the tool and a distal portion of the tool, the elongate section sized for passage through a sheath. The tool also includes jaws hinged at a first joint, the first joint located at the distal portion of the tool, the jaws adapted to vary between an open state and a closed state, the jaws in the closed state forming a blunt tip, the blunt tip sized for passage through the sheath. Additionally, the tool includes a handle located at the proximal portion of the tool, the handle including a first member and a second member hinged at a second joint and a lock, the lock adapted for releasably locking to the sheath. In one embodiment, the handle and the jaws are connected so that motion of the first handle member toward the second handle member moves the jaws from the closed state to the open state.

In additional embodiments, the present subject matter includes a tool, with an elongate section disposed between a proximal portion of the tool and a distal portion of the tool. Additionally, the tool includes movable jaws located at the distal portion of the tool, and a handle located at the proximal portion of the tool, the handle connected to the jaws and adapted to move the jaws between an open state and a closed state. Also, varying embodiments of the tool include a tool lock at the proximal portion of the tool and a sheath mateable to the tool and having a sleeve sized for at least partial passage of the elongate portion of tool and the jaws, the sheath having a proximal portion and a distal portion, the sleeve having a wall defining a slot extending longitudinally along the sleeve. Additional embodiments of the tool include a sheath catch at the proximal portion of the sheath, the sheath catch mateable to the tool lock.

Varying additional embodiments of the present subject matter include a method for implanting a device in a patient, including inserting a stylet at least partially into a sheath, opening an elongate canal in a patient using the fastened stylet and sheath, removing the stylet from the sheath, the sheath at least partially disposed in the elongate canal, inserting a tool in the sheath, the tool including a proximal portion and a distal portion, the proximal portion of the tool having a graspable handle adapted to manipulate jaws located at the distal portion between an open state and a closed state, creating a space in the patient through opening and closing the jaws through manipulation of the graspable handle, and movement of the fastened stylet and sheath, removing the tool from the sheath and removing the sheath from the elongate canal.

Varying examples of the present subject matter also include a method for implanting a device in a patient, including inserting a stylet at least partially into a sheath, releasably fastening the stylet to the sheath, opening an elongate canal adjacent a body lumen in a patient using the fastened stylet and sheath, removing the stylet from the sheath, the sheath at least partially disposed in the elongate canal, inserting a tool in the sheath, the tool including a proximal portion and a distal portion, the proximal portion of the tool having a graspable handle adapted to manipulate jaws located at the distal portion between an open state and a closed state, releasably fastening the tool to the sheath, creating a space in the patient through opening and closing the jaws through manipulation of the graspable handle, and movement of the fastened stylet and sheath, removing the tool from the sheath, guiding an implantable device into the sheath, the implantable device having an expandable element, directing the expandable element through the sheath and at least partially beyond the sheath, and into the space, providing a flowable material into the implantable device to at least partially restrict the body lumen, and removing the sheath.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

FIG. 1A illustrates a front view of a stylet, according to one embodiment of the present subject matter;

FIG. 1B illustrates a top view of a stylet, according to one embodiment of the present subject matter;

FIG. 1C illustrates a top view of a stylet lock, according to one embodiment of the present subject matter;

FIG. 2A illustrates a front view of a sheath, according to one embodiment of the present subject matter;

FIG. 2B illustrates a top view of the sheath, according to one embodiment of the present subject matter;

FIG. 2C illustrates a side view of the sheath, illustrating the first face, according to one embodiment of the present subject matter;

FIG. 2D illustrates a cross section taken at line 2D--2D in FIG. 2B;

FIG. 3A illustrates a perspective view of a tool, according to one embodiment of the present subject matter;

FIG. 3B is a close up of the jaws 306 illustrated in FIG. 3A;

FIG. 4A represents a front view of a tool in a first mode of operation, according to one embodiment of the present subject matter;

FIG. 4B illustrates a front view of a tool in an additional mode of operation, according to one embodiment of the present subject matter;

FIG. 5 is a longitudinal cross-sectional view of an implantable device, according to one embodiment of the present subject matter;

FIG. 6 is a cross-sectional view taken along line 6--6 of FIG. 5;

FIG. 7 illustrates a perspective view of a tool combined with a sheath, in one embodiment of the present subject matter;

FIG. 8 illustrates a perspective view of the sheath and an implantable medical device, according to one embodiment of the present subject matter;

FIG. 9 illustrates a method for using a tool, according to one embodiment of the present subject matter.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

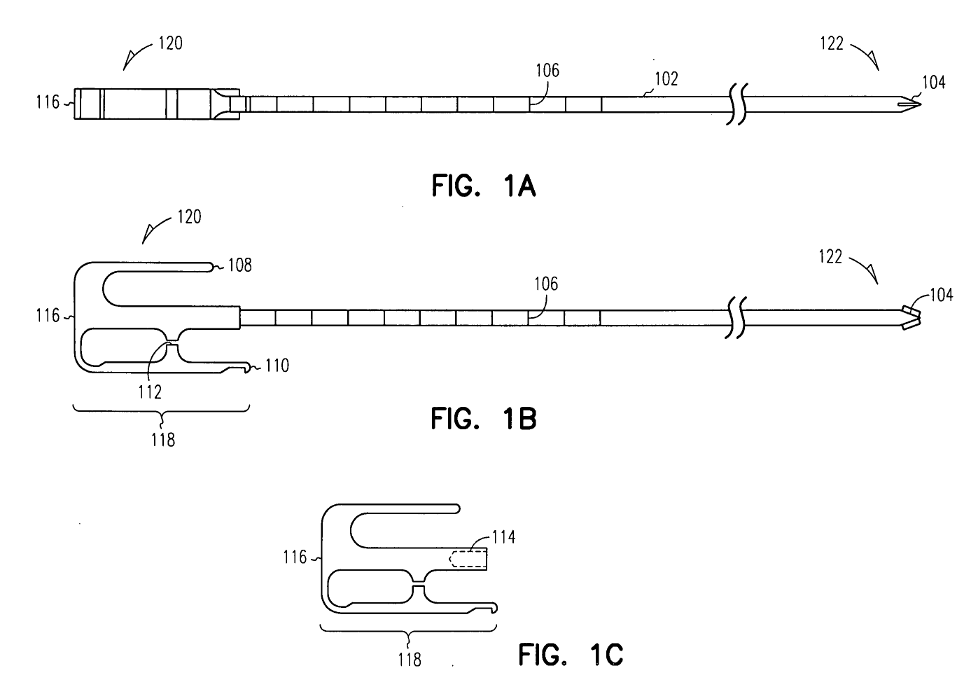

FIG. 1A illustrates a front view of a stylet, according to one embodiment of the present subject matter. In varying embodiments, the stylet includes a proximal portion 120 and a distal portion 122. The example illustrated includes a stylet lock 116 at the proximal portion 120, and an elongate portion 102 extending from the stylet lock 116 to the distal portion 122. Other configurations are possible without departure from the teachings of the present subject matter. In varying embodiments, the elongate portion 102 includes printed or engraved markings 106, which, in one embodiment, divide the elongate portion 102 axially into measuring units, such as centimeters. Generally, the stylet as illustrated is useful for opening an elongate canal in a body. Specifically, the stylet can be used for opening an elongate canal adjacent a body lumen in a patient.

Additionally, in varying embodiments, splines 104 are useful for tearing tissue. For example, twisting the stylet during creation of a channel in tissue can cause the splines 104 to wipe across various aspects of tissue. In varying embodiments, the splines include features, such as sharp edges, which grab tissue, and which can tear tissue, when wiped across areas of tissue in contact with the tip, or when used otherwise.

FIG. 1B illustrates a top view of a stylet, according to one embodiment of the present subject matter. In varying embodiments, the top view illustrates that one form of stylet lock 116 includes an alignment feature 108 and a locking barb 110, in addition to a flex-stop 112. In varying examples, the alignment feature 108 is useful for mating to a cavity adapted to receive the alignment feature 108, and in additional embodiments, the alignment feature 108 is useful as a register to stop axial movement of the stylet along a center axis of elongate portion 102.

In varying designs, the stylet lock 116 includes features which partially enable locking of the stylet with other equipment. For example, in one embodiment, the stylet lock 116 includes an elongate member 118 with a barb 110. The elongate member 118, in varying embodiments, elastically flexes relative to other portion of the stylet lock 116, and in one embodiment, the stylet lock includes a flex-stop 112 which is used to limit flexing of the elongate member 118. A flexible elongate member 118 is useful in varying embodiments to disengage the barb 110 from a mating feature, such as a catch. For example, in one embodiment, joining the stylet to other equipment elastically deforms the elongate member 118 temporarily. Further joining moves the barb 110 past a mating feature, releasing the stress which has previously deformed the elongate member 118. To release the stylet from other equipment, a user can manipulate the lock, inducing stress and causing elastic deformation of the elongate member 118 such that the barb clears any features in mating equipment, allowing for separation of the stylet from other equipment. In embodiments where a user grasps the stylet to disengage it from other equipment, the flex-stop 112 is useful to ensure that the operator does not introduce stress which permanently deforms the stylet lock 116.

The stylet, in varying designs, is useful for puncturing tissue. Tissue requires varying levels of pressure to become separated. By including features which increase the pressure applied to tissue to a level which separates tissue, the stylet can aid a user in creating a tissue pathway. For example, one embodiment of the stylet includes one or more splines 104 useful for increasing pressure and separating tissue. Additionally, the stylet includes varying tip designs at the distal portion 122. For example, in varying embodiments, the stylet includes a sharp tip, including, in varying embodiments, a pin-point design. In additional embodiments, the stylet includes a blunt tip, which is useful for creating a passageway in tissue while minimizing the risk of a user over-extending the depth of the canal.

It should be noted that the design enumerated in the discussion of exemplary illustration FIG. 1B performs various functions of the present subject matter, but is not exhaustive or exclusive in its representation of the designs within the scope of the present subject matter, and other embodiments are possible.

FIG. 1C illustrates a top view of a stylet lock, according to one embodiment of the present subject matter. In varying examples, the stylet includes a mating interface 114 useful for joining the stylet lock 116 to other components. In one example, an elongate member 118 useful for puncturing tissue is joined to the stylet lock 116. The mating interface 114 can include threads, or can join with other equipment using welds or permanent deformation of the stylet lock to the other equipment, a result achieved in one embodiment through crimping.

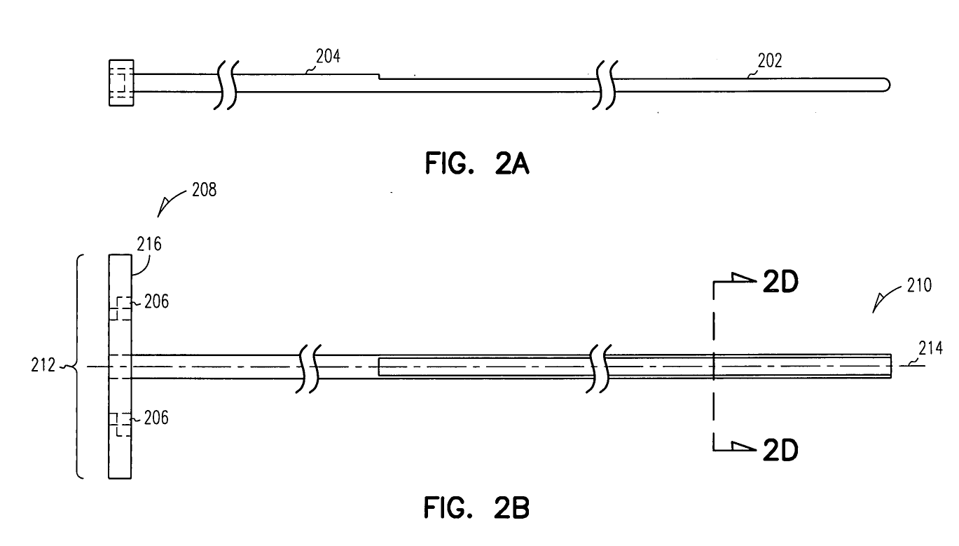

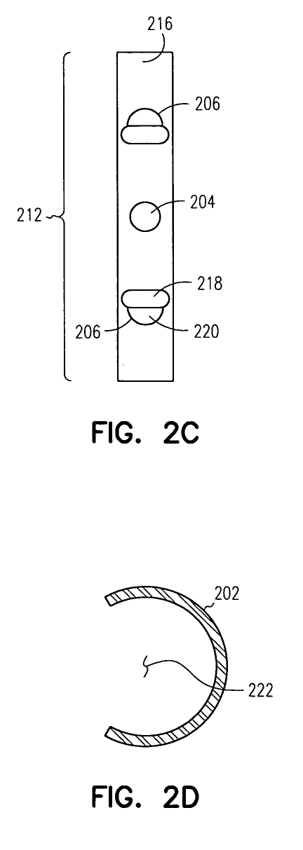

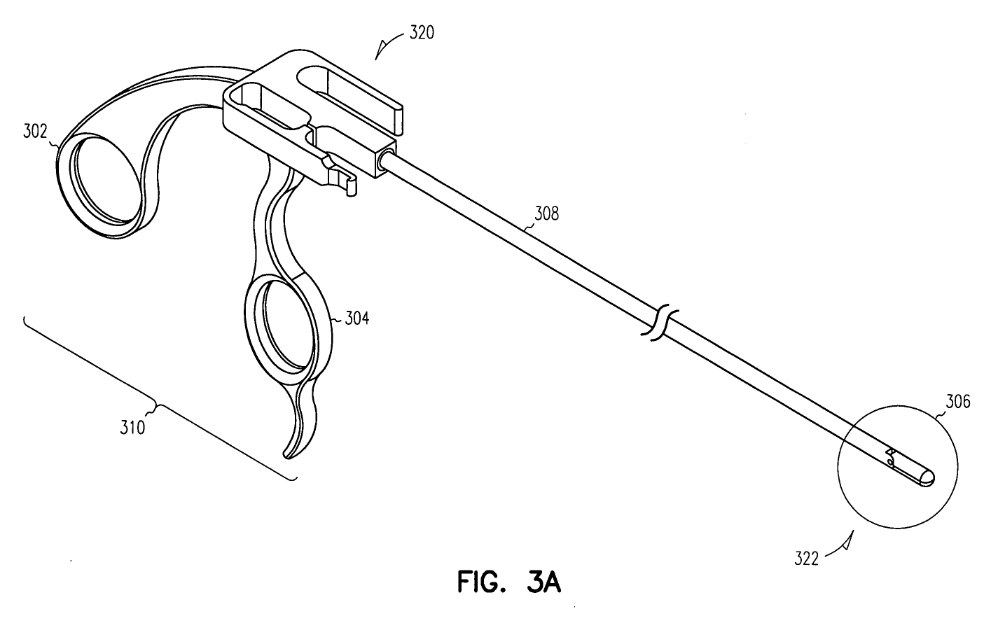

FIG. 2A illustrates a front view of a sheath, according to one embodiment of the present subject matter. In varying embodiments, the sheath includes a first section 204, and a second section 202, the first section constructed to fully encircle objects passing through it, such as the elongate portion of the stylet discussed in the example of FIGS. 1A-1B, and other objects. In addition to a first section which fully encircles an objects passing through it, or partially through it, the sheath includes a second section 202. In varying embodiments, the second section is adapted for partially encircling any objects passing through it.

Embodiments of the sheath including sections which encircle objects passing through a given portion, and further including sections which partially encircle objects passing through a given portion, are usefil in a number of ways. For example, by including a section which partially encircles objects, the sheath affords axis to the space defined by the portions which encircle the object, at varying points along the length of the sheath. In one example, the sheath is used to create a passageway in a medium. In varying embodiments, by at least partially retracting the sheath from the medium after creation of the passageway, a user may gain access to a passageway through access to at least a portion of the second section 202. In varying embodiments, an object intended for placement in the passageway of the medium can be placed in the space defined by the second section 202, and then inserted into the passageway. An additional benefit of the multi-section sheath design in that tools used for guiding one or more objects intended for placement in the passageway can be directed through the fully enclosed section 204.

It should be noted that a sheath which includes some structure which fully encircles an object passing through the structure benefits from improved rigidity under both bending and axial compressive stress. Additional rigidity under both bending and axial stress is useful to reduce the thickness and density of materials used to create the sheath, without hampering the sheath's ability to define spaces in various mediums. For example, in one embodiment, the sheath is used to at least partially define a passageway into a mammal body. Definition of a passageway into a mammal body requires the cutting of tissue and the displacement of body matter, and performance of these activities can put the sheath under bending and axial stress.

Additionally, portions of a sheath which includes structure which fully encircles objects passing through the structure are useful to minimize the flow of substances into the space defined by the sheath.

FIG. 2B illustrates a top view of the sheath, according to one embodiment of the present subject matter. In varying examples, the sheath includes a proximal portion 208, a distal portion 210, and a first face 216. In varying embodiments, the sheath includes a sheath catch, 206. The sheath catch is useful for fastening the sheath to other objects, and in some embodiments is useful for releasably fastening the sheath to other objects. Additionally, in some embodiments, the sheath catch includes portions which are graspable 212. The graspable portion can be used to aid in the creation or maintenance of a passageway in a medium, in various embodiments.

Depending on which examples are used, the sheath includes a symmetrical catch design which eases the alignment requirements of the sheath with other objects. For example, the sheath includes a catch, which in one embodiment is offset from center axis 214. It should be noted that the present subject matter includes sheaths having non-circular cross sections in addition to the illustrated circular cross sections. In the embodiments where the catch is to one side of the center axis 214, additional aspects, such as mirroring mechanical interfaces, are provided to increase the positional flexibility of the sheath with respect to objects to which it is fastened. In other words, in one example, the sheath can be fastened to an object using a mechanical connection on a first side of the center axis 214, and the sheath can additionally be fastened to the same object using a mechanical connection on a second side, when rotated approximately 180 degrees around the center axis 214.

It is of note that the illustration shows one example, and in the example the catch is illustrated as a single contact point, but that overall, the subject matter encompasses embodiments where the catch includes two or more contact points between the sheath and additional objects.

FIG. 2C illustrates a side view of the sheath, illustrating the first face, according to one embodiment of the present subject matter. Directly visible is a first face 216, as well as other aspects of the embodiment. For example, catch 206 is visible, which, in varying embodiments, includes a channel 218, and a plateau 220. The channel 218, in some examples, passes through the graspable portion 212. The plateau 220, in varying embodiments, is defined by a plane which is approximately parallel to the first face 216, but which is sunk into the graspable portion 212. In varying embodiments, a lock which is sized for passage through the channel 218 is disposed in the channel 218, and includes a feature adapted to mate with the plateau 220. For example, in one embodiment, a barb passes through the channel 218 and hooks the plateau. In the embodiment pictured, the lock passing through the channel must be sized to allow lateral motion of the lock with respect to center axis of channel 218 and the plateau 220, the flexing of the lock enabling passage of the barb through the channel 218.

FIG. 2D illustrates a cross section of the sheath pictured in FIG. 2B, according to one embodiment of the present subject matter. In varying embodiments, the sheath includes a second section 202 which is circle shaped, and only partially encircles a space 222, which, in varying embodiments, is sized for passage of an object. Although the cross section shows a circular shape, other shapes are within the scope of the present subject matter, such as partially closed rectangles, and other shapes.

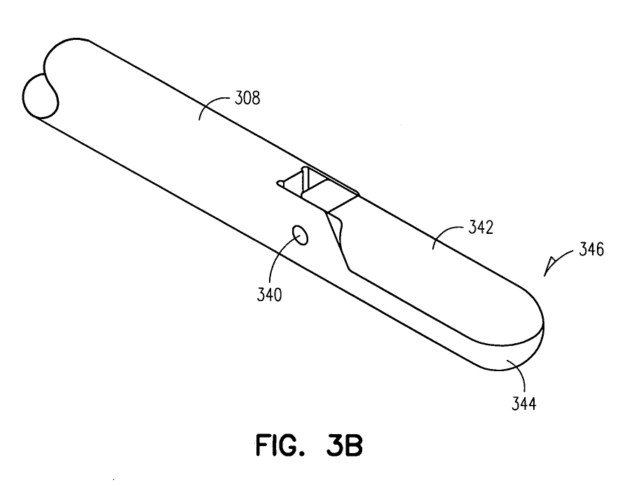

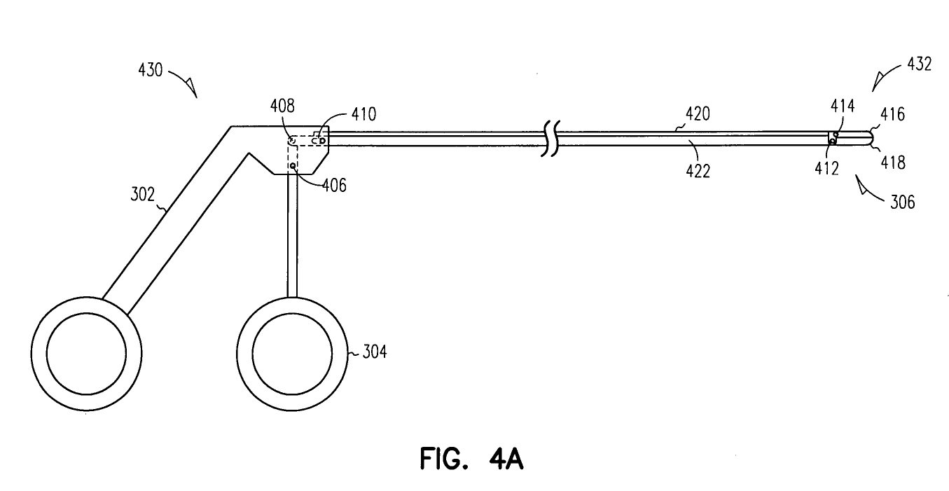

FIG. 3A illustrates a perspective view of a tool, according to one embodiment of the present subject matter. In varying embodiments, the tool includes a graspable handle 310 located at proximal portion 320. The graspable handle, in varying embodiments, includes a static handle member 302 and a pivot handle member 304. Additionally, the tool includes jaws 306, and an elongate shaft 308 disposed between the graspable handle 310 and the jaws. In varying embodiments, pivoting the pivot handle 304 opens and closes the jaws 306. In varying designs, this is accomplished through linkage mechanisms. One mechanism uses a four-bar linkage.

FIG. 3B is a close up of the jaws 306 illustrated in FIG. 3B, according to various embodiments of the present subject matter. In varying embodiments, the jaws include a static jaw 344 which is statically fixed to the elongate shaft 308. Additionally, varying embodiments include a pivot jaw 342 which is movably mounted to the elongate shaft 308. In varying embodiments, the pivot jaw is mounted to the elongate shaft using a pin 340 which is pressed into a cavity in the elongate shaft 308, and which is further pressed into a cavity in the pivot jaw 342. In some of these embodiments, the pin 340 is blended to match the contour of the elongate shaft 308. In other embodiments, the pin 340 is a boss which extends away form the pivot jaw 342. In some of these embodiments, the pin 340 and the pivot jaw 342 are a product of the same workpiece. In varying embodiments, the pivot jaw 342 rotates with respect to elongate shaft 308. In additional embodiments, other joints are used. Examples of other joints include a sliding joint, or a ball-in-socket joint.

In varying embodiments, the pivot jaw 342 and the static jaw 344 are configured for mating. For example, the static jaw 344 and the pivot jaw 342, in one embodiment, are shaped to smoothly extend the envelope defined by the elongate shaft 308. In varying embodiments, the jaws can be mated to form a blunt tip 346. Other embodiment include jaws mated to form a point, or to form a shape with one or more edges useful for cutting mammalian tissue.

In varying embodiments, through movement of the pivot jaw, the static jaw and the pivot jaw either contact each other or are near to each other. In varying examples, the portions of the pivot jaw and the static jaw which either contact or pass near to each other include serrated features, such as knurling and ridges, which populate all or a portion of the areas which are in contact or are proximal each other. For example, in one embodiment, the pivot jaw is shaped like one half a cylinder, and the static jaw is shaped like one half a cylinder, and the portions of each jaw which are not curved include features such as knurling, which populate all or a portion of the non-curved structure of the respective jaw. In one embodiment, the pivot jaw includes features dimensioned to interlock with features on the static jaw. In an additional example, the static jaw and the fixed jaw move into a mated position forming an aperture. For example, in one embodiment, the pivot jaw is one half a toroidal shape, and the static jaw is one half a toroidal shape, and the mated pair define an aperture.

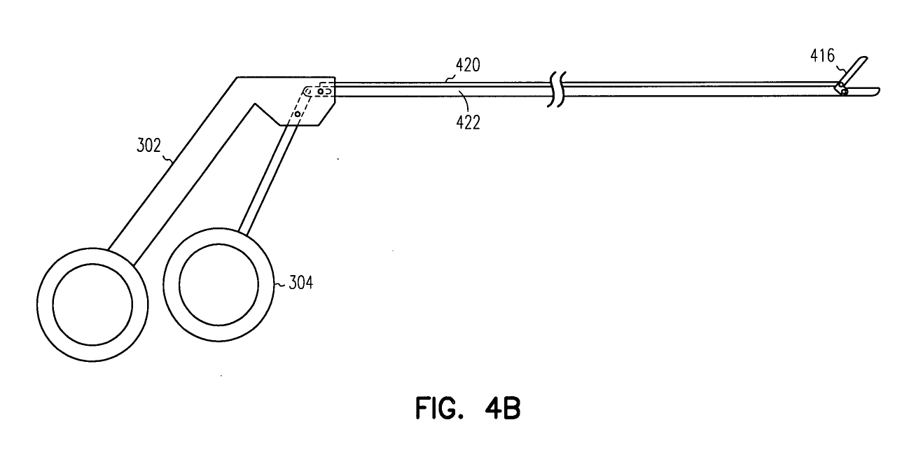

FIGS. 4A-4B represent varying aspects of a linkage capable of performing one function of the present subject matter, according to one embodiment of the present subject matter.

FIG. 4A represents a front view of a tool in a first mode of operation, according to one embodiment of the present subject matter. The illustration includes a proximal portion 430 and a distal portion 432.

The tool of the present description includes a static handle 302 and a pivot handle 304. In varying embodiments, a user can grasp at once, with a single normal hand, both the static handle and the pivot handle, to open and close the jaws 306. In varying embodiments, this function is enabled by a four-bar linkage, the four-bar linkage including the pivot handle 304, the linkage 422, the elongate shaft 420, and the pivot jaw 416. For example, by moving the pivot handle approximately laterally toward the proximal portion of the tool, a user can increase the distance between the pivot jaw 416 and the static jaw 418. Operating the tool has a tendency to move the linkage 422 laterally toward the distal portion, but this motion is provided in this embodiment for illustration only, and other mechanical relationships connecting pivot handle motion to pivot jaw motion are within the scope of the present invention.

Focusing on the pictured embodiment, it is apparent that the function is enabled by a series of elements which rotate about various joints. For example, a handle pivot joint 406 serves as a connection point between the elongate shaft 420 and the pivot handle 302. Additionally, in varying embodiments, the static handle 302 is fixed to the elongate shaft, and the handle pivot joint serves to fix the pivot handle to the structure to which the static handle is a component. In varying embodiments, the handle pivot joint does not move laterally. The pivot handle-to-linkage joint 408 serves to fix the pivot handle to the linkage 422, allowing pivot handle 304 rotation with respect to linkage 422. During operation of the tool, the pivot handle-to-linkage joint moves laterally, in varying embodiments. In one embodiment, the joint travels on an axis which is approximately parallel to the elongate shaft 420, but other embodiments are within the scope of the present subject matter.

The linkage-to-pivot jaw joint 414 allows rotation of the pivot jaw 416 with respect to the linkage 422, in varying embodiments of the present subject matter. In some embodiments, the linkage-to-pivot joint moves laterally along an axis which is substantially parallel to the elongate shaft 420.

In varying examples, a pivot jaw joint 412 serves as a connection point between the elongate shaft 420 and the pivot jaw 416. Additionally, in varying embodiments, the static jaw 418 is fixed to the elongate shaft, and the handle pivot joint serves to fix the pivot handle to the structure to which the static handle is a component. In varying embodiments, the pivot jaw joint 412 does not move laterally.

The pivot jaw joint 412 serves to fix the pivot jaw 416 to the linkage 422, allowing pivot jaw 416 rotation with respect to linkage 422. During operation of the tool, the pivot jaw joint moves laterally, in varying embodiments. In one embodiment, the joint travels on an axis which is approximately parallel to the elongate shaft 420, but other embodiments are within the scope of the present subject matter.

In one embodiment, because of the proximity of the linkage 422 to the elongate shaft 420, the linkage-to-pivot jaw joint includes a joint which allows either the pivot jaw to slide respective to the linkage, the pivot handle to slide respective of the linkage, or both the pivot handle and the pivot jaw to slide respective to the handle. Sliding can be accomplished using varying mechanical constructs, including a slot and a pin, elastic deformation, or other forms of translation. While these embodiments are useful for teaching aspects of the present subject matter, they are not exhaustive or exclusive, and should not be interpreted as limiting.

FIG. 4B illustrates a front view of a tool in an additional mode of operation, according to one embodiment of the present subject matter. It should be noted that, in various embodiments, the nature of the movements of the tool is suited for the stresses associated with creating a space in a medium. For example, in some embodiments, if the movement of the pivot handle were in another direction, the tool would not capitalize on natural strengths of the users as the tool was manipulated in a patient.

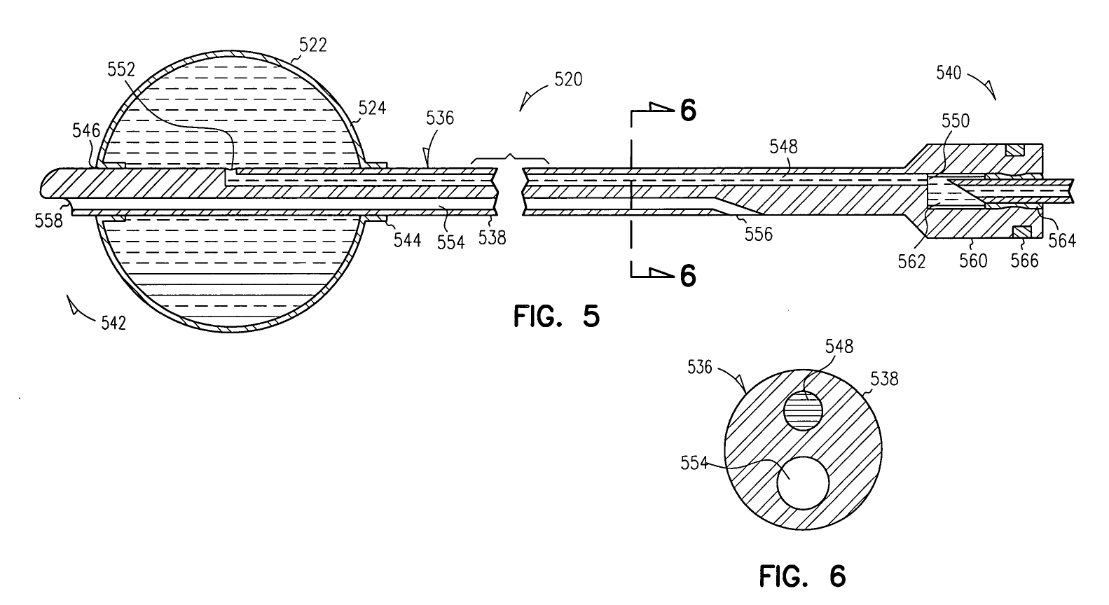

Referring now to FIGS. 5-6 there is shown one embodiment of the implantable device 520. In varying embodiments, the implantable device includes a tubular elongate body 536, where the tubular elongate body 536 has a peripheral surface 538, a proximal portion 540 and a distal portion 542. The adjustable element 522 has at least one opening through the continuous wall 524 to which the peripheral surface 538 is connected to and sealed to the adjustable element 522. The tubular elongate body 536 is inserted through the first opening 544 and the second opening 546 such that the distal portion 542 of the elongate body 536 partially extends beyond the outer surface 532 of the adjustable element 522. The peripheral surface 538 is then sealed to the walls creating the first opening 544 and the second opening 546. In one embodiment, the peripheral surface 38 is sealed to the openings using a chemical or polymer adhesive, such as silicone. In an alternative embodiment, the peripheral surface 538 is sealed to the openings using sonic welding techniques as are known in the art.

The tubular elongate body 536 further includes a first interior passageway 548 extending longitudinally in the tubular elongate body 536 from a first port 550 at the proximal portion 540 to a second port 552 in fluid communication with the chamber 528 of the implantable device for adjustably expanding or contracting the expandable element 522 by applied fluid volume introduced through the first port 550.

In an additional embodiment, the tubular elongate body 536 further includes a second interior passageway 554 extending longitudinally in the tubular elongate body 536 from a proximal opening 556 through the peripheral surface 538 positioned between the proximal portion 540 and the distal portion 542. In some embodiments, the passageway 554 extends to a distal opening 558 located in the distal portion 542 of the tubular elongate body 536. In one embodiment, the second interior passageway 554 is of sufficient diameter to receive and guide a guidewire for the insertion of the implantable device 520 into a human body. In additional embodiments, the second interior passageway 554 is sized for a stylet. In an alternative embodiment, the guidewire is removably attached at the distal portion 542 and passes outside the expandable element 522 and along side the elongate body 536 to allow placement of the device without the need for the second interior passageway 554.

The implantable device 520 further includes a rear port element 560 coupled to the proximal portion 540 of the tubular elongate body 536. In one embodiment, the rear port element 560 is coupled to the proximal portion 540 of the elongate body 536 using chemical adhesives, or alternatively, using sonic welding techniques as are known in the art. In an additional embodiment, the rear port element 560 and proximal portion 540 are formed together in a polymer extrusion process or polymer casting process as are known in the art.

The rear port element 560 includes a cavity 562, where the cavity 562 is in fluid communication with the first port 550 of the elongate body 536. The rear port element 560 also includes an elastic septum 564 through which the cavity 562 is accessed. The elastic septum 564 is retained in the rear port element 560 by a clamp ring 566 located around the rear port element 560. In one embodiment, the clamp ring 566 is made of a biocompatible material, such as, for example, titanium. In one embodiment, the elastic septum 564 is made of a biocompatible material, such as, for example, silicone or polyurethane.

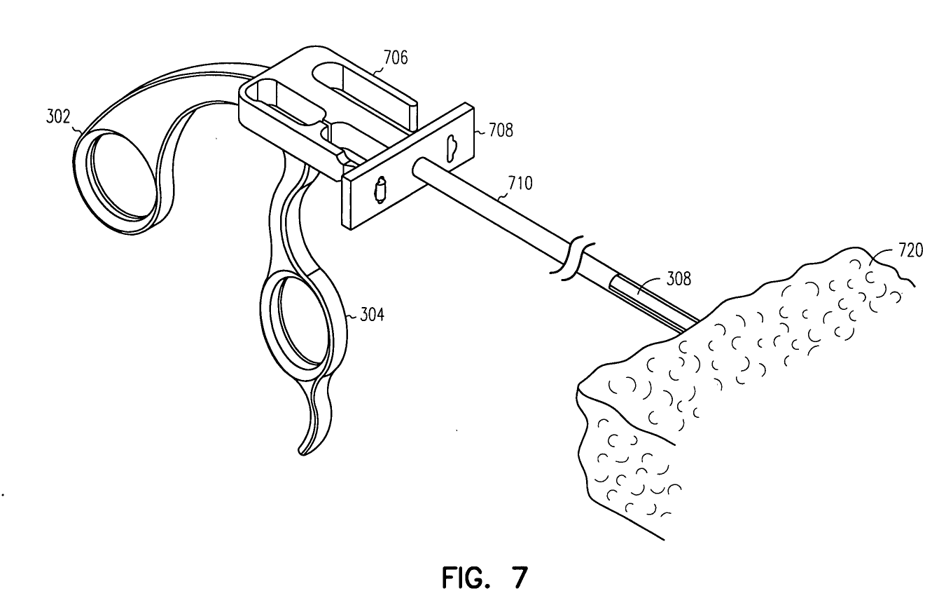

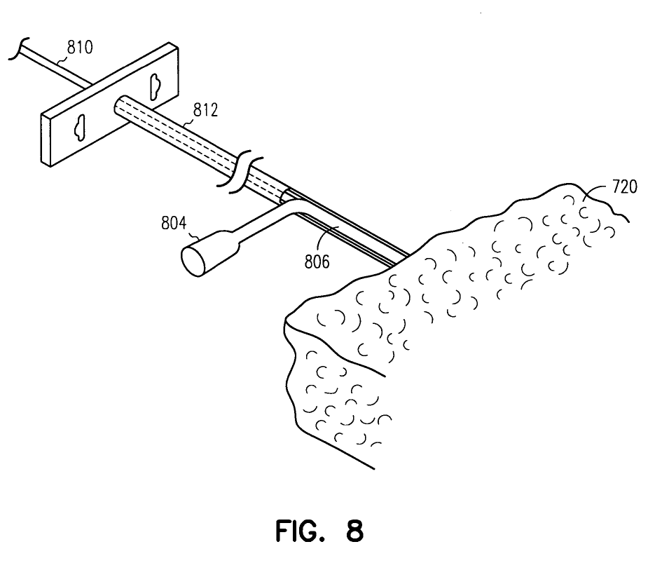

FIG. 7 illustrates a perspective view of a tool combined with a sheath, in one embodiment of the present subject matter. In varying embodiments, the tool includes a lock 706, a static handle 302, a pivot handle 304, and an elongate shaft 308. Additionally illustrated is a sheath including a catch 708, and a conduit 710. In varying embodiments, the tool and the sheath are releasably fastened to one another.

In varying embodiments, the sheath is disposed in a medium 720. In one embodiment, the sheath was moved into position in the medium 720 using a stylet such as the stylet of FIGS. 1A-1C, however other methods of positioning the sheath in a medium 720 are within the scope of the present subject matter. In varying embodiments, the tool is combined with the sheath which is disposed in the medium 720. The combined tool and sheath are useful for creating a space in the medium 720. For example, in varying embodiments, the tool is moved from the first mode of operation illustrated in the example of FIG. 4A to the second mode of operation illustrated in the example of FIG. 4B, creating a space in the medium 720.

FIG. 8 illustrates a perspective view of the sheath and an implantable medical device, according to one embodiment of the present subject matter. In varying embodiments, an implantable medical device 804 includes an elongate portion 806 which is placed in the sheath 812, and is positioned in the sheath 812 for implantation in a medium 720. In varying embodiments, the device is similar to the device discussed in the teachings associated with FIGS. 5-6, however other implantable devices are within the scope of the present subject matter.

In varying embodiments, implantation is aided by the assistance of a guidewire 810. However, in additional embodiments, the implantable device is adapted for use with a stylet, such as the stylet discussed in the teachings of FIGS. 1A-1B. Still other devices within the scope of the present subject matter are useful for implanting the implantable device 804, such as a tool, or other devices. In varying embodiments, devices used during implantation of the implantable device, such as the guidewire 810, are disposed in the sheath approximately coaxial with the center line of the sheath. However, various embodiments are within the scope of the present subject matter, including embodiment using elements for aiding in implanting an implantable medical device which pass through openings in any passageways, including open portions of a sheath, and in canals located in the patient.

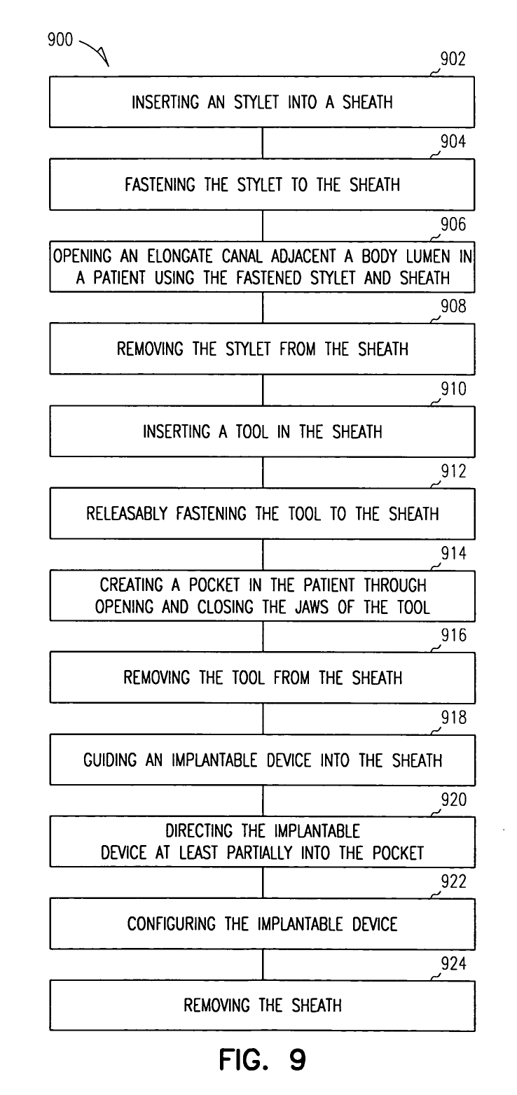

FIG. 9 illustrates a method for using a tool, according to one embodiment of the present subject matter. In varying embodiment, one method includes inserting a stylet at least partially into a sheath 902. Additionally, various embodiments include releasably fastening the stylet to the sheath 904, and, in various examples, opening an elongate canal adjacent a body lumen in a patient using the fastened stylet and sheath 906. Also, varying embodiments includes removing the stylet from the sheath 908, while the sheath is at least partially disposed in the elongate canal. In varying additional embodiments, the present subject matter also includes inserting a tool in the sheath 910, the tool including a proximal portion and a distal portion, the proximal portion of the tool having a graspable handle adapted to manipulate jaws at the distal portion. In various examples, the present subject matter includes releasably fastening the tool to the sheath 912, and, in varying embodiments, creating a space in the patient 914 through opening and closing the jaws through manipulation of the graspable handle, and movement of the fastened stylet and sheath. Various embodiments include removing the tool from the patient 916. Also, in varying examples, the present subject matter includes guiding an implantable device into the sheath 918, the implantable device having an expandable element, and, in varying examples, directing the expandable element through the sheath and at least partially beyond the sheath, and into the space 920. Additionally, the present subject matter includes providing a flowable material into the implantable device to at least partially restrict the body lumen 922, and, in varying embodiments, removing the sheath 924.

Varying locations for incision fall within the scope of the present subject matter. For example, in one embodiment, an incision is made in the perineum, through which a canal is created extending proximal a body lumen, such as the urethra. In additional embodiments, an incision is made in the labia, through which a canal is created extending proximal a body lumen, such as the urethra. Placing an expandable element in a canal, such as those previously disclosed, and expanding the element, restricts the body lumen, in varying embodiments of the present subject matter.

Still further variations of the present subject matter include varying aids useful for placement of the sheath, the stylet, the tool, the expandable element, and other aspects, in varying embodiments of the present subject matter. For example, in one embodiment, the sheath includes an anomaly in shape, such as a depression, which can be recognized using x-ray, or other forms of patient inspection. Additionally, the sheath can include a contrast agent, such as fluoroscopic tint, useful for identification during fluoroscopy. The use of a shape anomaly, or fluoroscopic tint is also contemplated with the stylet, the tool, the expandable element, and other aspects of the system and methods disclosed here.

Although specific embodiments have been illustrated and described herein, it will be appreciated by those of ordinary skill in the art that any arrangement which is calculated to achieve the same purpose may be substituted for the specific embodiment shown. This application is intended to cover adaptations or variations of the present subject matter. It is to be understood that the above description is intended to be illustrative, and not restrictive. Combinations of the above embodiments, and other embodiments will be apparent to those of skill in the art upon reviewing the above description. The scope of the present subject matter should be determined with reference to the appended claims, along with the full scope of equivalents to which such claims are entitled.

We claim:

1. A tool, comprising:
an elongate section disposed between a proximal portion of the tool and a distal portion of the tool;
jaws hinged at a first joint, the first joint located at the distal portion of the tool, the jaws adapted to vary between an open state and a closed state; and
a handle located at the proximal portion of the tool, the handle including a first member and a second member hinged at a second joint and a lock, the jaws adapted to move from the closed state to the open state when the first handle member is moved toward the second handle member.

2. The apparatus of claim 1, wherein the tool includes a four bar linkage.

3. The apparatus of claim 2, wherein the connected handle and jaws are adapted to move the jaws from the open state to the closed state when the first handle member is moved away from the second handle member.

4. The apparatus of claim 2, wherein the jaws are at least partially knurled.

5. The apparatus of claim 4, wherein the elongate portion is substantially cylindrical.

6. A system, comprising:
a tool, including:
an elongate section disposed between a proximal portion of the tool and a distal portion of the tool;
movable jaws located at the distal portion of the tool;
a handle located at the proximal portion of the tool, the handle connected to the jaws and adapted to move the jaws between an open state and a closed state; and
a tool lock at the proximal portion of the tool, and
a sheath mateable to the tool, the sheath including:
a sleeve sized for at least partial passage of the elongate portion of tool and the jaws, the sheath having a proximal portion and a distal portion, the sleeve having a wall defining a slot extending longitudinally along the sleeve; and
a sheath catch at the proximal portion of the sheath, the sheath catch mateable to the tool lock.

7. The apparatus of claim 6, wherein the tool includes a four bar linkage.

8. The apparatus of claim 7, wherein the jaws have a pin-point tip.

9. The apparatus of claim 7, wherein the jaws have a blunt tip.

10. A method for implanting a device in a patient, comprising:
inserting a stylet at least partially into a sheath;
opening an elongate canal in a patient using the fastened stylet and sheath,
removing the stylet from the sheath, the sheath at least partially disposed in the elongate canal;
inserting a tool in the sheath, the tool including a proximal portion and a distal portion, the proximal portion of the tool having a graspable handle adapted to manipulate jaws located at the distal portion between an open state and a closed state;
creating a space in the patient through opening and closing the jaws through manipulation of the graspable handle, and movement of the fastened stylet and sheath;
removing the tool from the sheath; and
removing the sheath from the elongate canal.

11. The method of claim 10, further comprising releasably locking the tool into the sheath.

12. The method of claim 10, wherein a first stylet with a sharp tip is used to open the elongate canal.

13. The method of claim 12, further comprising expanding the space by twisting the stylet.

14. The method of claim 12, wherein a second stylet with a dull tip is used to open the elongate canal.

15. The method of claim 12, further comprising expanding the space by moving the sheath and the stylet into and out of the patient at least once.

16. The method of claim 10, further comprising guiding at least one implantable device into the sheath, the at least one implantable device having an expandable element.

17. The method of claim 16, further comprising directing the expandable element through the sheath and at least partially beyond the sheath, and partially into the space.

18. The method of claim 17, further comprising guiding the implantable device with a guidewire.

19. The method of claim 16, further comprising adjusting the at least one implantable device.

20. The method of claim 19, further comprising providing a flowable material into the implantable device to at least partially restrict a body lumen of the patient.
